(19) United States
(12) Patent Application Publication
(10) Pub. No.: US 2009/0192438 A1
(43) Pub. Date: Jul. 30, 2009

(54) Use of Iontophoresis or Electrotherapy and Ultrasound to Deliver Melanin for Skin Rejuvenation

(76) Inventor: Dennis R. Jones, Chesapeake, VA (US)

Correspondence Address: Dennis R. Jones, 1025 Executive Blvd., Ste: 112, Chesapeake, VA, 23320, US

(21) Appl. No.: 12/358,775

(22) Filed: Jan. 23, 2009

Related U.S. Application Data
(60) Provisional application No. 61/023,526, filed on Jan. 25, 2008.

(51) Int. Cl. A61M 37/00; A61N 1/30

(52) U.S. Cl. 604/20; 604/22

(57) ABSTRACT

The method disclosed includes the combining of iontophoresis or electrotherapy and ultrasound to simultaneously deliver the appropriate chromophore to the skin for cosmetic or therapeutic treatment of the skin and skin conditions by the use of a laser, Intense Pulsed Light (IPL), a combination of laser and Radio Frequency (RF) energy, or a combination of IPL and RF.

### INVENTION DISCLOSURE

This application claims priority to Provisional Application No. 61/023,526, and a filing date of Jan. 25, 2008. This application contains no new matter.

### FIELD OF INVENTION

The method disclosed includes the combining of iontophoresis or the electrotherapy and ultrasound to simultaneously deliver the appropriate chromophore to the skin for cosmetic or therapeutic treatment of the skin and skin conditions by the use of a laser, Intense Pulsed Light (IPL), a combination of laser and Radio Frequency (RF) energy, or a combination of IPL and RF. The treatments include but are not limited to: skin rejuvenation, collagen remodeling, the reduction or removal of fine lines wrinkles, skin tightening, skin lifting, and shrinking the skin following liposuction.

### BACKGROUND OF INVENTION

Historically, there have been a number of options for cosmetic or therapeutic treatment of the skin by the use of a laser, Intense Pulsed Light (IPL), a combination of laser and Radio Frequency (RF) energy, or a combination of IPL and RF. However, the treatments include the use of a light source that targets a chromophore residing in the skin.

Problems exist with the current techniques, however. The success for the treatments listed above is in proportion to the availability of the targeted chromophore residing in the skin, which can vary greatly from patient to patient.

The use of a light source as a method of treating various pathological and cosmetic conditions, particularly skin rejuvenation, can be divided into two general methods. The first being essentially the application of laser or light source energy selected to be absorbed preferentially by elements residing naturally within the skin. The second being differentiated from the first by introduction of a foreign element to provide an effective radiation target with an absorption frequency equal to or different from the naturally occurring elements in the skin.

### SUMMARY OF THE INVENTION

This application claims priority to Provisional Application No. 61/023,526, and a filing date of Jan. 25, 2008. This application contains no new matter. It is an objective of the present disclosure to provide a novel and improved method of the treatment of the skin by employing iontophoresis or electrotherapy and ultrasound or High Intensity Focused Ultrasound (HIFU) simultaneously or in conjunction with transdermal delivery of agents that may be delivered in pure form, in solution, as emulsions, within liposomes, within synthetic or natural microspheres, polymeric nanopaiticles, microparticles, microcapsules, or other known micro encapsulation vehicles. Agents may include, without limitation, natural or synthetic melanin.

Agents may be delivered in pure form, in solution, in emulsions, in liposomes, in synthetic or natural microspheres, polymeric nanoparticles, microparticles, microcapsules, or other known micro encapsulation and include a topical gel, lotion, liquid, cream or ointment composition that may also include melanin encapsulated liposomes. The agent is designed to attach to, or become physically incorporated into the skin.

The ultrasound energy causes break up of the top most surface of the skin and allows the melanin encapsulated liposome composition to migrate into the skin. This technique preferentially ultra-exposes the skin to light energy which is absorbed preferentially by naturally occurring and synthetic elements in skin. Laser or other intense pulsed light between 100 and 3000 nm is appropriate for targeting melanin naturally occurring and synthetic elements that are being delivered.

The combined use of melanin encapsulated liposomes in conjunction with iontophoresis or electrotherapy and ultrasound or iontophoresis or electrotherapy and HIFU followed by a treatment using a laser, IPL, a combination of laser and RF energy, or a combination of IPL and RF allows for immediate treatments such as skin rejuvenation, collagen remodeling, reduction or removal of fine lines wrinkles, skin tightening, skin lifting, and shrinking the skin following liposuction on selected locations on the body of mammals to be performed.

It is another object of the disclosure to provide a composition where the liposomes have a size range of 1 nanometer (nm) to 500 microns. However, a diameter between manometer and 10 microns is preferred.

It is another object of the disclosure to provide an improved method of skin rejuvenation, collagen remodeling, the reduction or removal of fine lines wrinkles, skin tightening, skin lifting, and shrinking the skin following liposuction by the use of IPL, a combination of laser and RF energy, a combination of IPL and RF or other light energy of a wavelengths or multiple wavelengths which are absorbed by the melanin delivered to the skin by the process of ultrasound and electrical energy delivered simultaneously.

It is a further object of the disclosure that a variety light energy sources, both coherent and incoherent, including such lasers as ruby red and Nd-YAG, alexandrite, and diode lasers, or any other light source providing an effective frequency and intensity.

It is also another object of the disclosure that the light energy should be in the wavelength range of 100 to 3000 nm.

It is also another object of the disclosure that the pulse length is from about 1 nanoseconds to about 1000 milliseconds.

1. A method of providing skin rejuvenation, collagen remodeling, the reduction or removal of fine lines wrinkles, skin tightening, skin lifting, and shrinking the skin following liposuction by employing a combination of iontophoresis or electrotherapy and ultrasound, IPL and laser or equivalent light energy, wherein said combination includes using iontophoresis or electrotherapy and ultrasound or HIFU simultaneously for transdermally delivering natural or synthetically derived melanin by using a topical gel, lotion, liquid, cream or ointment formulation, said method also comprising melanin encapsulated liposomes using ultrasound for causing break up of the top most surface of the skin allowing said melanin encapsulated liposomes composition or other chromophores to migrate into said skin such that said liposome composition or other chromophores may reside in or become physically incorporated into the skin so that using IPL, laser, a combination of laser and RF, a combination of IPL and RF or equivalent light energy results in preferential ultra-exposure of said hair follicles providing focus for photothermolysis to facilitate the skin treatments mentioned.

2. The method of claim 1, wherein skin treatments can be provided in one or more patient treatments.

3. The method of claim 1, wherein said skin treatments is provided in a single treatment.

4. The method of claim 1, wherein melanin delivered to the skin is the chromophore for the skin treatments.

5. The method of claim 1, wherein a laser or other intense pulsed light between 300 to 3000 nm is appropriate for targeting melanin in the skin.

6. The method of claim 1, wherein removal employing a combination of, iontophoresis or electrotherapy, ultrasound or HIFU, and laser or equivalent light energy eliminates the need to continuously prepare and re-prepare the skin with other drugs or chromophores.

7. The method of claim 1, comprising the step of exposing the skin to at least one enzyme prior to exposure to ultrasound and iontophoresis or electrotherapy.

8. The method of claim 1, wherein said pulse length for laser light is from about 1 nanosecond to about 1 second.

9. The method of claim 1, wherein said pulse length is from about 1 nanoseconds to about 1000 milliseconds,

10. The method according to claim 1, wherein the laser or equivalent light energy fluence received at said hair producing tissue is 300 J/cm² or less.

11. The method according to claim 1, wherein the energy fluence received at said hair producing tissue is greater than 1 J/cm² and further comprises cooling said tissue.

12. The method according to claim 1, wherein said energy fluence received at said tissue is from about from about 1×10⁻⁶ J/cm² to 1 J/cm².

13. The method of claim 1, wherein said energy fluence received at said tissue is from about from about 1×10⁻³ J/cm² to about 0.1 J/cm².

14. The method of claim 1, further comprising cooling said tissue to maintain a temperature of said tissue below a threshold for thermal injury.

15. The method of claim 1, further comprising the step of maintaining the temperature of said tissue at or below 38 degrees Celsius.

16. A set of devices for providing permanent hair removal employing a combination of, iontophoresis or electrotherapy and ultrasound, IPL and laser or equivalent light energy, wherein said combination includes using iontophoresis or electrotherapy and ultrasound or HIFU simultaneously for transdermally delivering natural or synthetically derived melanin, by using a topical gel, lotion, liquid, cream or ointment formulation, said method also comprising melanin encapsulated liposomes using ultrasound for causing break up of the top most surface of the skin allowing said melanin encapsulated liposomes composition or other chromophores to migrate into said skin such that said liposome composition or other chromophores may reside in or become physically incorporated into the skin so that using IPL, laser, a combination of laser and RF, a combination of IPL and RF or equivalent light energy devices results in preferential ultra-exposure of said skin providing focus for the aforementioned skin rejuvenation treatments.

17. The devices of claim 16, wherein melanin is the skin chromophore for the selective skin rejuvenation and wherein said laser device or other intense pulsed light between 100 to 3000 nm is appropriate for targeting melanin in a hair follicle shaft.

18. The devices of claim 16 wherein said pulse length for laser light from said laser device is from about 1 nanosecond to about 1 secondhand more preferably wherein said pulse length is from about 1 nanoseconds to about 300 milliseconds.

19. The devices according to claim 16, wherein the laser or equivalent light energy fluence received at said hair producing tissue is 300 J/cm² or less, preferably greater than 1 J/cm² and further comprises cooling said tissue and most preferably from about 1×10⁻⁶ J/cm² to 1 J/cm².
